(19) United States
(12) Patent Application Publication
(10) Pub. No.: US 2023/0140143 A1
(43) Pub. Date: May 4, 2023

(54) Behavior Modification Feedback For Improving Diabetes Management

(71) Applicant: Dexcom, Inc., San Diego, CA (US)

(72) Inventors: Giada Acciaroli, San Diego, CA (US); Margaret A. Crawford, San Diego, CA (US); Mark Derdzinski, San Diego, CA (US); Lauren H. Jepson, San Diego, CA (US); Sarah Kate Pickus, San Diego, CA (US); Robert J. Dowd, San Diego, CA (US); Apurv U. Kamath, San Diego, CA (US)

(73) Assignee: Dexcom, Inc., San Diego, CA (US)

(21) Appl. No.: 17/974,290

(22) Filed: Oct. 26, 2022

Related U.S. Application Data
(60) Provisional application No. 63/263,188, filed on Oct. 28, 2021.
(60) Provisional application No. 63/292,957, filed on Dec. 22, 2021.

(51) Int. Cl. G16H 40/63; G16H 20/00

(52) U.S. Cl. CPC G16H 40/63; G16H 20/00

(57) ABSTRACT

Glucose measurements are received and features for corresponding time periods over a time window are generated, the features being values indicating whether the user has been engaging in beneficial diabetes management behaviors. Using the aggregated features patterns indicating that beneficial diabetes management behaviors are not being engaged in are identified. Potential behavior modification feedback is generated by including in the potential behavior modification feedback at least one behavior modification feedback, for each of the identified patterns, that a user could take to engage in beneficial diabetes management behavior. At least one of the potential behavior modification feedback is selected and displayed or otherwise presented to the user.

### RELATED APPLICATIONS

This application claims the benefit of U.S. Provisional Patent Application No. 63/292,957, filed Dec. 22, 2021, and titled “Behavior Modification Feedback For Improving Diabetes Management,” the entire disclosure of which is hereby incorporated by reference, and also claims the benefit of U.S. Provisional Patent Application No. 63/263,188, filed October 28, 2021, and titled “Ranking Feedback For Improving Diabetes Management,” the entire disclosure of which is hereby incorporated by reference.

### BACKGROUND

Diabetes is a metabolic condition affecting hundreds of millions of people and is one of the leading causes of death worldwide. For people living with Type I diabetes, access to treatment is critical to their survival and it can reduce adverse outcomes among people with Type II diabetes. With proper treatment, serious damage to the heart, blood vessels, eyes, kidneys, and nerves due to diabetes can be avoided. Regardless of the type of diabetes (e.g., Type I or Type II), managing diabetes successfully involves monitoring and oftentimes adjusting food and activity to control a person's blood glucose, such as to reduce severe fluctuations in and/or generally lower the person's glucose.

However, many conventional glucose monitoring applications employ user interfaces that display raw glucose information in a manner that is difficult for users to interpret, particularly users who have just recently started monitoring their glucose. Consequently, users may be unable to draw insights from the data and thus are unable to alter their behavior in a meaningful way in order to improve their glucose. Furthermore, over time these users often become overwhelmed and frustrated by the manner in which information is presented by these conventional glucose monitoring applications and thus discontinue use of these applications before improvements in their glucose and overall health can be realized. Moreover, as users increasingly utilize mobile devices (e.g., smart watches and smart phones) to access glucose monitoring information, the failure by conventional systems to provide meaningful glucose information in a manner that users can act upon is further exacerbated by the constraints imposed by the small screens of these mobile devices.

### SUMMARY

To overcome these problems, techniques for behavior modification feedback for improving diabetes management are discussed. In one or more implementations, in a continuous glucose level monitoring system glucose measurements are obtained, for each time window in multiple time windows, for a user for a first time period of multiple time periods in the window. One or more features for the first time periods of the multiple time windows are generated from the glucose measurements. A pattern in the glucose measurements in the first time periods of the multiple time windows is detected from the one or more features for the first time periods of the multiple time windows. A behavior modification feedback to improve glucose levels corresponding to the pattern is determined, a user interface including the behavior modification feedback is generated, and the user interface is caused to be displayed.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example of an implementation that is operable to implement behavior modification feedback for improving diabetes management as described herein.

FIG. 2 depicts an example of an implementation of a wearable glucose monitoring device in greater detail.

FIG. 3 is an illustration of an example architecture of a behavior modification identification system.

FIG. 4 illustrates an example of providing behavior modification recommendations for improving diabetes management.

FIG. 5 illustrates an example of sizes of normalized sizes for different detected patterns.

FIG. 6 depicts a procedure in an example of implementing behavior modification feedback for improving diabetes management.

FIG. 7 illustrates an example of a system generally that includes an example of a computing device that is representative of one or more computing systems and/or devices that may implement the various techniques described herein.

### DETAILED DESCRIPTION

Overview

Techniques for behavior modification feedback for improving diabetes management are discussed herein. Broadly, blood glucose level measurements of a user are obtained over time. Glucose level measurements can be obtained by a wearable glucose monitoring device being worn by the user. These glucose level measurements can be produced substantially continuously, such that the device may be configured to produce the glucose level measurements at regular or irregular intervals of time (e.g., approximately every hour, approximately every 30 minutes, approximately every 5 minutes, and so forth), responsive to establishing a communicative coupling with a different device (e.g., when a computing device establishes a wireless connection with a wearable glucose level monitoring device to retrieve one or more of the measurements), and so forth. These glucose level measurements are analyzed to identify behavior modifications for the user to make in order to improve their diabetes management.

In one or more implementations, a data stream of glucose measurements is received. One or more features for a particular time period are generated and stored, each feature being a value that can be computed from the glucose measurements and that indicates whether the user has been engaging in beneficial diabetes management behaviors. The features may include metrics that are a representation or summarization of the data in the data stream for a particular time period. These time periods are, for example, different multi-hour blocks of time during a day. E.g., a day may include a first time period from midnight to 6 am (corresponding to sleep or night), a second time period from 6 am to noon (corresponding to after breakfast), a third time period from noon to 6 pm (corresponding to after lunch), and a fourth time period from 6 pm to midnight (corresponding to after dinner). These time periods may be fixed or may be adaptively identified based on various received data (e.g., sleep onset may be detected by an activity monitor and may be used to determine the beginning of the “sleep” time period on that date, user input may specify beginning or ending times for a time period (e.g., user input received via a user preferences user interface displayed to the user), and so forth).

The features for a time period are aggregated over a time window, such as one week. These aggregated features are used to identify patterns indicating that beneficial diabetes management behaviors are not being engaged in. For example, one feature may be a time in range feature (e.g., the range being glucose levels between 70 milligrams per deciliter (mg/dL) and 180 mg/dL) and a pattern indicating that beneficial diabetes management behaviors are not being engaged in may be that the time in range for a particular time period over a week is less than 70%. Potential behavior modification feedback is generated, for each of the identified patterns, that a user could take to engage in beneficial diabetes management behavior. At least one of the potential behavior modification feedback is selected and displayed or otherwise presented to the user.

Behavior modification feedback, also referred to as an actionable goal, refers to one or more actions that the user can take to alter (e.g., improve) his or her diabetes management. Examples of behavior modifications include “Take an evening walk 3 times this week,” “Eat a dinner low in carbohydrates 2 nights this week,” “To not eat close to bedtime, try setting a time that you will stop eating after each evening,” and so forth.

The techniques discussed herein generate behavior modification feedback for improving diabetes management and provide notifications of such to the user. This provides goals or behavior modification feedback to the user in a way that is informative and actionable for the user to improve their health, longevity, diabetes management, and so forth. This can allow the user to make appropriate changes in their lifestyle, reducing the need to monitor their glucose levels closely if they follow the behavior modification feedback.

Furthermore, the techniques discussed herein allow goals or suggestions of how to improve diabetes management to be generated and presented to the user. Thus, rather than (or in addition to) simply displaying raw glucose data, the techniques discussed herein allow useful actions or steps to take to be identified to the user so that they can improve their diabetes management without having to try to figure out how to do so based on the raw glucose data alone.

In the following discussion, an example environment is first described that may employ the techniques described herein. Examples of implementation details and procedures are then described which may be performed in the example environment as well as other environments. Performance of the example procedures is not limited to the example environment and the example environment is not limited to performance of the example procedures.

Example of an Environment

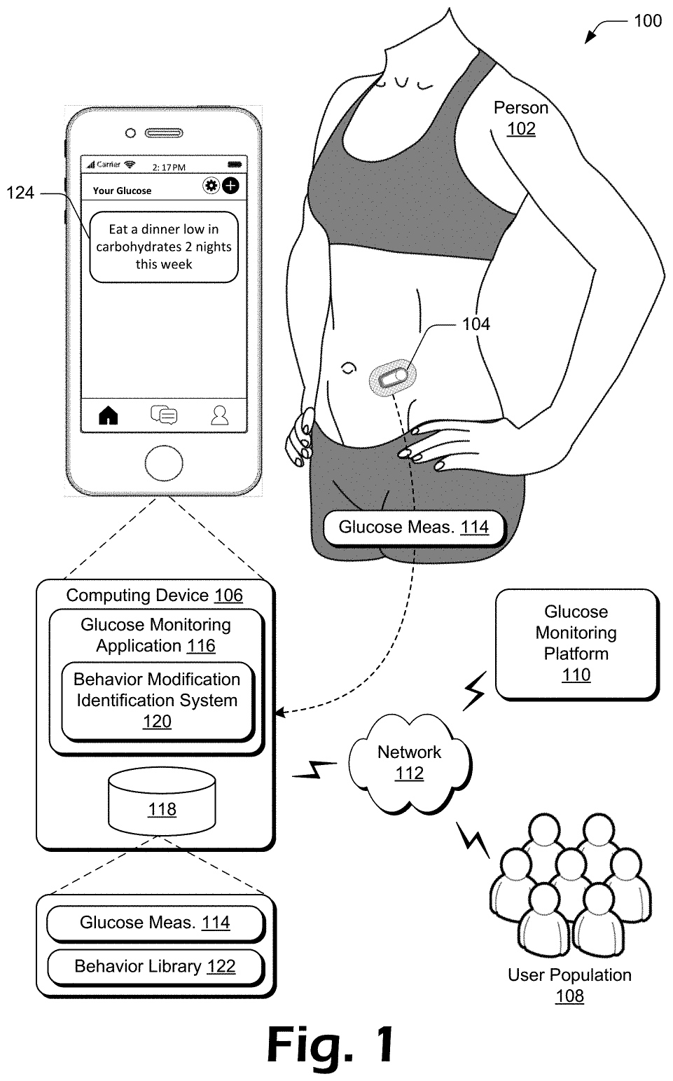

FIG. 1 is an illustration of an environment 100 in an example of an implementation that is operable to implement feedback for improving diabetes management as described herein. The illustrated environment 100 includes a person 102, who is depicted wearing a wearable glucose monitoring device 104. The illustrated environment 100 also includes a computing device 106, other users in a user population 108 that wear glucose monitoring devices 104, and a glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102's glucose. Although a wearable glucose monitoring device is discussed herein, it is to be appreciated that user interfaces for glucose monitoring may be generated and presented in connection with other devices capable of providing glucose measurements, e.g., non-wearable glucose devices such as blood glucose meters requiring finger sticks, patches, and so forth. In implementations that involve the wearable glucose monitoring device 104, though, it may be configured with a glucose sensor that continuously detects analytes indicative of the person 102's glucose and enables generation of glucose measurements. In the illustrated environment 100 and throughout the detailed description these measurements are represented as glucose measurements 114.

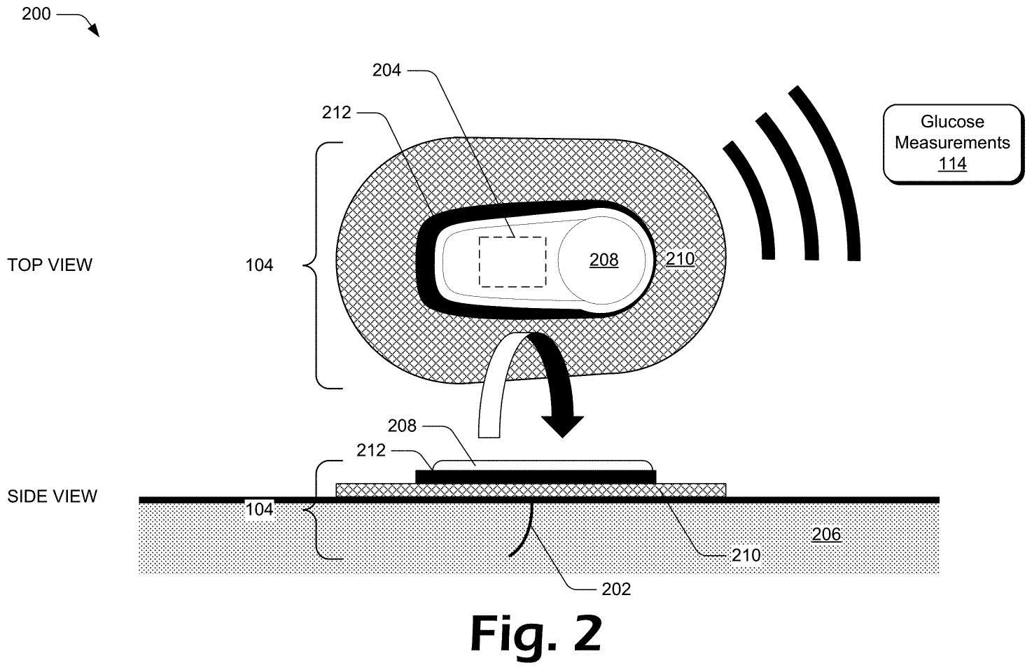

In one or more implementations, the wearable glucose monitoring device 104 is a continuous glucose monitoring (“CGM”) system. As used herein, the term “continuous” used in connection with glucose monitoring may refer to an ability of a device to produce measurements substantially continuously, such that the device may be configured to produce the glucose measurements 114 at regular or irregular intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth. This functionality along with further aspects of the wearable glucose monitoring device 104's configuration is discussed in more detail in relation to FIG. 2.

Additionally, the wearable glucose monitoring device 104 transmits the glucose measurements 114 to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements 114 to the computing device 106 at set time intervals. For example, the wearable glucose monitoring device 104 may be configured to communicate the glucose measurements 114 to the computing device 106 every five minutes (as they are being produced).

Certainly, an interval at which the glucose measurements 114 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 of the person 102 at least temporarily, e.g., in computer-readable storage media of the computing device 106.

Although illustrated as a mobile device (e.g., a mobile phone), the computing device 106 may be configured in a variety of ways without departing from the spirit or scope of the described techniques. By way of example and not limitation, the computing device 106 may be configured as a different type of device, such as a mobile device (e.g., a wearable device, tablet device, or laptop computer), a stationary device (e.g., a desktop computer), an automotive computer, and so forth. In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 from the wearable glucose monitoring device 104, communicate them via the network 112 to the glucose monitoring platform 110, display information related to the glucose measurements 114, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to the glucose measurements 114. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the discussed techniques, the computing device 106 is configured to implement behavior modification feedback for improving diabetes management. In the environment 100, the computing device 106 includes glucose monitoring application 116 and storage device 118. Here, the glucose monitoring application 116 includes the behavior modification identification system 120. Although illustrated as being included in computing device 106, additionally or alternatively at least some functionality of the behavior modification identification system 120 is located elsewhere, such as in glucose monitoring platform 110. Further, the glucose measurements 114 and a behavior library 122 are shown stored in the storage device 118. The storage device 118 may represent one or more databases and also other types of storage capable of storing the glucose measurements 114 and the behavior library 122. The behavior library 122 stores multiple behavior modifications that can be provided to the user 102, for example that a user could take to engage in beneficial diabetes management behavior and likely improve his or her glucose levels.

In one or more implementations, the glucose measurements 114 and/or the behavior library 122 may be stored at least partially remote from the computing device 106, e.g., in storage of the glucose monitoring platform 110, and retrieved or otherwise accessed in connection with configuring and outputting (e.g., displaying) user interfaces for diabetes management feedback presentation. For instance, the glucose measurements 114 and/or the behavior library 122 may be generally stored in storage of the glucose monitoring platform 110 along with the glucose measurements of the user population 108 and/or the behavior library 122, and some of that data may be retrieved or otherwise accessed on an as-needed basis to display user interfaces for diabetes management feedback presentation.

Broadly speaking, the glucose monitoring application 116 is configured to support interactions with a user that allow behavior modifications to improve the user's diabetes management to be presented. This may include, for example, obtaining the glucose measurements 114 for processing (e.g., to determine the appropriate behavior modifications), receiving information about a user (e.g., through an onboarding process and/or user feedback), causing information to be communicated to a health care provider, causing information to be communicated to the glucose monitoring platform 110, and so forth.

In one or more implementations, the glucose monitoring application 116 also leverages resources of the glucose monitoring platform 110 in connection with behavior modification feedback for improving diabetes management. As noted above, for instance, the glucose monitoring platform 110 may be configured to store data, such as the glucose measurements 114 associated with a user (e.g., the person 102) and/or users of the user population 108, and the behavior library 122. The glucose monitoring platform 110 may also provide updates and/or additions to the glucose monitoring application 116. Further still, the glucose monitoring platform 110 may train, maintain, and/or deploy algorithms (e.g., machine learning algorithms) to generate or select feedback or to identify time periods for which feedback is provided, such as by using the wealth of data collected from the person 102 and the users of the user population 108. One or more such algorithms may require an amount of computing resources that exceeds the resources of typical, personal computing devices, e.g., mobile phones, laptops, tablet devices, and wearables, to name just a few. Nonetheless, the glucose monitoring platform 110 may include or otherwise have access to the amount of resources needed to operate such algorithms, e.g., cloud storage, server devices, virtualized resources, and so forth. The glucose monitoring platform 110 may provide a variety of resources that the glucose monitoring application 116 leverages in connection with enabling diabetes management feedback to be presented via user interfaces.

In accordance with the described techniques, the behavior modification identification system 120 is configured to use the glucose measurements 114 to identify one or more behavior modifications in the behavior library 122 and cause output of one or more user interfaces that present the identified one or more behavior modifications. The glucose monitoring application 116 may cause display of the configured user interface 124 via a display device of the computing device 106 or other display device.

As discussed above and below, a variety of behavior modifications may be selected or generated based on the glucose measurements 114 of the user in accordance with the described techniques. In the context of measuring glucose, e.g., continuously, and obtaining data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, user interfaces including diabetes management feedback presentation may be configured and displayed (or otherwise output) in connection with other types of devices for glucose monitoring, such as non-wearable devices (e.g., blood glucose meters requiring finger sticks), patches, and so forth.

In this example 200, the wearable glucose monitoring device 104 is illustrated to include a sensor 202 and a sensor module 204. Here, the sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user initiated application of the wearable glucose monitoring device 104 is nearly painless and does not require the withdrawal of blood. Moreover, the automatic sensor applicator generally enables the person 102 to embed the sensor 202 subcutaneously into the skin 206 without the assistance of a clinician or health care provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the sensor 202 is communicatively coupled to the sensor module 204 via at least one communication channel which can be a wireless connection or a wired connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of diabetes management using one or more measurement techniques. In one or more implementations, the sensor 202 may also be configured to detect analytes in the blood or the interstitial fluid that are indicative of other markers, such as lactate levels, which may improve accuracy in generating various diabetes management feedback. Additionally or alternately, the wearable glucose monitoring device 104 may include additional sensors to the sensor 202 to detect those analytes indicative of the other markers.

In another example, the sensor 202 (or an additional sensor of the wearable glucose monitoring device 104 — not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples, the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 114 based on the communications with the sensor 202 that are indicative of the above-discussed changes. Based on these communications from the sensor 202, the sensor module 204 is further configured to generate communicable packages of data that include at least one glucose measurement 114. In one or more implementations, the sensor module 204 may configure those packages to include additional data, including, by way of example and not limitation, a sensor identifier, a sensor status, temperatures that correspond to the glucose measurements 114, measurements of other analytes that correspond to the glucose measurements 114, and so forth. It is to be appreciated that such packets may include a variety of data in addition to at least one glucose measurement 114 without departing from the spirit or scope of the described techniques.

In implementations where the wearable glucose monitoring device 104 is configured for wireless transmission, the transmitter 208 may transmit the glucose measurements 114 wirelessly as a stream of data to a computing device. Alternately or additionally, the sensor module 204 may buffer the glucose measurements 114 (e.g., in memory of the sensor module 204 and/or other physical computer-readable storage media of the wearable glucose monitoring device 104) and cause the transmitter 208 to transmit the buffered glucose measurements 114 later at various intervals, e.g., time intervals (every second, every thirty seconds, every minute, every five minutes, every hour, and so on), storage intervals (when the buffered glucose measurements 114 reach a threshold amount of data or a number of measurements), and so forth.

Having considered an example of an environment and an example of a wearable glucose monitoring device, consider now a discussion of some examples of details of the techniques for behavior modification feedback for improving diabetes management.

System Architecture

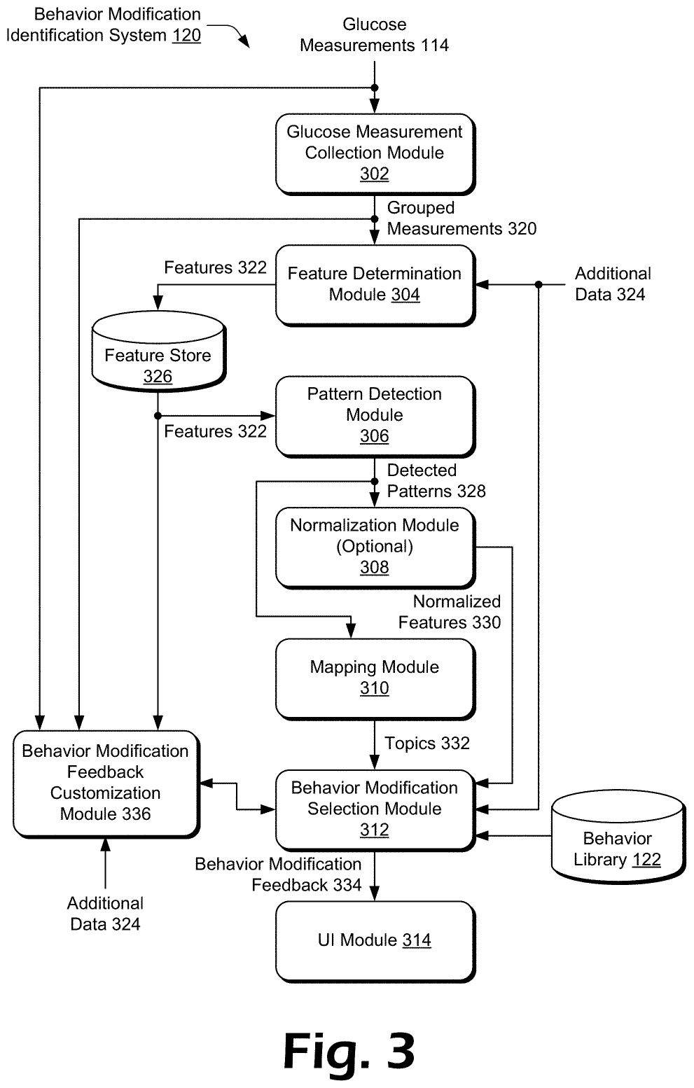

FIG. 3 is an illustration of an example architecture of a behavior modification identification system 120. The behavior modification identification system 120 includes a glucose measurement collection module 302, a feature determination module 304, a pattern detection module 306, a normalization module 308, a mapping module 310, a behavior modification selection module 312, and a UI module 314. Generally, the behavior modification identification system 120 analyzes the glucose measurements 114 for the user 102 and looks for patterns in the glucose measurements 114 that indicate poor (or non-optimal) diabetes management by the user. Poor diabetes management can be quantified in various manners, such as glucose measurements 114 not staying within a particular range, glucose measurements 114 varying by greater than a particular amount, and so forth. In one or more implementations, the behavior modification identification system 120 identifies poor diabetes management by identifying patterns in glucose measurements 114 for a given time period of a time window across multiple time windows (e.g., for a given multi-hour time period, such as 6 am to noon, on each of multiple days).

The glucose measurement collection module 302 receives glucose measurements 114 and optionally timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. The glucose measurement collection module 302 groups the glucose measurements 114 into different time periods referred to as grouped measurements 320.

In one or more implementations, each time period is a portion of a day (or other 24 hour interval). These time periods are chosen to capture the impacts of specific diabetes management decisions and lifestyle choices. In one or more implementations, each day is separated into multiple time periods based on when the user eats meals and when the user sleeps. For example, a day may include a first time period from midnight to 6 am (corresponding to sleep or night), a second time period from 6 am to noon (corresponding to after breakfast), a third time period from noon to 6 pm (corresponding to after lunch), and a fourth time period from 6 pm to midnight (corresponding to after dinner). Additionally or alternatively, additional time periods can correspond to other user actions that affect glucose levels, such as when the user exercises.

The glucose monitoring application 116 optionally provides a user interface via which the user 102 can customize the time periods to his or her typical schedule. For example, assume the user 102 typically goes to bed at 10 pm, eats breakfast at 7 am, eats lunch at noon, and eats dinner at 5 pm. These times can be provided to the glucose monitoring application 116 (e.g., by the user), which determines the time periods for the day to include a first time period from 10 pm to 7 am (corresponding to sleep or night), a second time period from 7 am to noon (corresponding to after breakfast), a third time period from noon to 5 pm (corresponding to after lunch), and a fourth time period from 5 pm to midnight (corresponding to after dinner). A day may be separated into other numbers of periods than four. For example, assume the user 102 typically goes to bed at 10 pm, exercises at 5 am, eats breakfast at 7 am, eats lunch at 11 am, eats an afternoon snack at 2 pm, and eats dinner at 6 pm. These times can be provided to the glucose monitoring application 116, which determines the time periods for the day to include a first time period from 10 pm to 5 am (corresponding to sleep or night), a second time period from 5 am to 7 am (corresponding to exercise), a third time period 7 am to 11 am (corresponding to after breakfast), a fourth time period from 11 am to 2 pm (corresponding to after lunch), a fourth time period from 2 pm to 6 pm (corresponding to snack), and a sixth time period from 6 pm to 10 pm (corresponding to after dinner).

Additionally or alternatively, different time periods for the user 102 can be automatically learned by the glucose monitoring application 116 by monitoring various data available to the glucose monitoring application 116 (e.g., exercise or sleep patterns from an activity tracker, eating patterns from a food or calorie tracking application) or detected directly (e.g., sleep onset detected by activity tracker). Various rules or criteria can be used to determine time periods based on the various data available to the glucose monitoring application 116, such as detecting sleep onset and sleep cessation from an activity tracker and using the times of sleep onset and sleep cessation to determine the time period corresponding to sleep.

In one or more implementations, the glucose monitoring application 116 uses a machine learning system to determine the different time periods for the user 102. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple data (e.g., times of exercise, sleep, or eating during a day) and timestamps indicating when the exercise, sleep, or eating was done. Known labels are associated with the sets of multiple data indicating a time period that the data corresponds to. The machine learning system is trained by updating weights or values of layers in the machine learning system to minimize the loss between time periods generated by the machine learning system for the training data and the corresponding known labels for the training data. Various different loss functions can be used in training the machine learning system, such as cross entropy loss, mean squared error loss, and so forth.

In one or more implementations the machine learning system is trained over time as the glucose monitoring application 116 is used over time. E.g., the user can provide an indication of whether a particular time period is correct, and this indication can be used as a known label for the current time periods and used to further train the machine learning system.

Accordingly, different time periods can be established for different users. Furthermore, different time periods can be established for different days. For example, the user 102 may have different schedules on different types of days (e.g., a different schedule on weekends and holidays than on weekdays, a different schedule on days the user works than on days the user does not work). Accordingly, the time periods for different types of days can be provided by the user 102 or determined by a machine learning system of the glucose monitoring application 116.

In one or more embodiments, the blocks of times for different time periods can vary for a user across different days. For example, a user may typically go to sleep between 11 pm and midnight, and wake up between 5:30 am and 6:30 am. For any given day, the time the user goes to sleep and the time the user wakes up can be detected using various data streams, such as data from an activity tracker worn by the user. Accordingly, the time period corresponding to sleep for the user may be 11:13 pm to 6:00 am for one day, 11:27 pm to 5:48 am the next day, 11:45 pm to 6:12 am the next day, and so forth.

The feature determination module 304 generates one or more features 322 based on the grouped measurements 320. A feature 322 refers to any value that can be computed from the glucose measurements 114 (and optionally additional data) and that indicates whether the user has been engaging in beneficial diabetes management behaviors or lifestyle choices. A feature 322 can be a metric that is a representation or summarization of the data in the glucose measurements 114 or for a particular time period during the time window.

In one or more implementations, the feature determination module 304 also receives additional data 324. The additional data 324 refers to any additional data that may be used to identify poor diabetes management. For example, the additional data 324 can include data that relates to user interactions with the computing device 106, with the display of the computing device 106, or with other system components that indicate level of engagement with diabetes management. E.g., this data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by behavior modification identification system 120, the number of times an application (e.g., glucose monitoring application 116) is viewed or launched, the timing of when an application (e.g., glucose monitoring application 116) is viewed or launched, the time spent reviewing glucose data or previous insights or educational materials, the frequency of interactions with coaches or clinicians, and so forth. Such data can be received from various sources, such as from the glucose monitoring application 116, from an operating system running on the computing device 106, from the glucose monitoring platform 110, and so forth. The additional data 324 may also include other data from other sources as discussed in more detail below.

In one or more implementations, each feature 322 is one or two values that represent or summarize the glucose measurements 114 or additional data 324 for a particular time period across the time window, transforming the glucose measurements 114 into a numeric indicator of the adherence to beneficial diabetes management and lifestyle choices. For example, each feature 322 can be a value that represents or summarizes the glucose measurements 114 across a week for the time periods corresponding to sleep during the week.

The feature determination module 304 generates, for corresponding time periods in a time window, any of a variety of features 322. In one or more implementations, the feature determination module 304 generates any of a variety of statistics from the glucose measurements 114, such as mean glucose measurement in the corresponding time periods, coefficient of variation for the glucose measurements in the corresponding time periods (the ratio of the standard deviation to the mean for the glucose measurements in the time periods), standard deviation of the glucose measurements in the time periods, receiver operating characteristics (ROC) and so forth.

Additionally or alternatively, the feature determination module 304 generates a time in range feature, such as an amount of time during the time periods the glucose measurements were in an acceptable or desired range of glucose levels, e.g., between 70 mg/dL and 180 mg/dL, or a narrow range between 70 mg/dL and 130 mg/dL. This acceptable or desired range can be a default range, can be a custom range set by the user or a health care professional, and so forth. Different time in range features having different acceptable or desired ranges of glucose levels can be generated for different corresponding time periods (e.g., the range of glucose levels for the time periods corresponding to sleep may be different than the range of glucose levels for the time periods corresponding to after lunch).

Additionally or alternatively, the feature determination module 304 generates a time above threshold feature, such as an amount of time during the time periods the glucose measurements were above a particular glucose level (e.g., 180 mg/dL or 250 mg/dL). This particular glucose level can be a default level, can be a custom level set by the user or a health care professional, and so forth. Multiple time above threshold features each having a different particular glucose level can be generated.

Additionally or alternatively, the feature determination module 304 generates a time below threshold feature, such as an amount of time during the time periods the glucose measurements were below a particular glucose level (e.g., 70 mg/dL). This particular glucose level can be a default level, can be a custom level set by the user or a health care professional, and so forth. Multiple time below threshold features each having a different particular glucose level can be generated.

Additionally or alternatively, the feature determination module 304 generates a maximum glucose measurement feature that is the maximum glucose measurement received during the time periods.

Additionally or alternatively, the feature determination module 304 generates a post-prandial feature, such as post-prandial glucose level peak, post-prandial area under the curve (AUC), an amount of post-prandial time the glucose measurements were above a particular glucose level (e.g., 250 mg/dl), and so forth.

Additionally or alternatively, the feature determination module 304 generates a fasting glucose in range feature, such as an indication (e.g., true or false) indicating whether a particular glucose measurement was in an acceptable or desired range of glucose levels, e.g., between 70 milligrams per deciliter (mg/dL) and 180 mg/dL, or a narrow range between 70 mg/dL and 130 mg/dL. This acceptable or desired range can be a default range, can be a custom range set by the user or a health care professional, and so forth. Different time in range features having different acceptable or desired ranges of glucose levels can be generated for different corresponding time periods. For example, a fasting glucose in range feature can be generated based on a glucose measurement received just prior to the first food the user eats each morning, one of the last glucose measurements received at the end of the time periods corresponding to sleep, and so forth. By way of another example, a bedtime glucose in range feature can be generated based on a glucose measurement received at the beginning of the time period corresponding to sleep, and so forth.

Additionally or alternatively, the feature determination module 304 generates other features, such as maximum glucose measurement rate of change in the time periods, maximum glucose measurement rise in the time periods, low blood glucose index (LBGI) in the time periods, high blood glucose index (HBGI) in the time periods, a value indicating a rate of increase or decrease in glucose levels in the time periods, and so forth.

In one or more implementations, the feature determination module 304 generates features from additional data 324, which can be various different types of data received from various different sources as discussed herein. For example, the feature determination module 304 can generate as features 322 a number of times the glucose monitoring application 116 was viewed or launched in the time periods, the number of times the glucose monitoring application 116 was launched or viewed after meals (e.g., at the beginning of time periods corresponding to after breakfast, after lunch, after dinner, etc.), and so forth.

The feature determination module 304 stores the generated features 322 in a feature store 326 (e.g., maintained on storage device 118). The generated features 322 are maintained for a duration time that can vary by implementation. For example, the generated features 322 may be maintained for two weeks, one month, one year, and so forth.

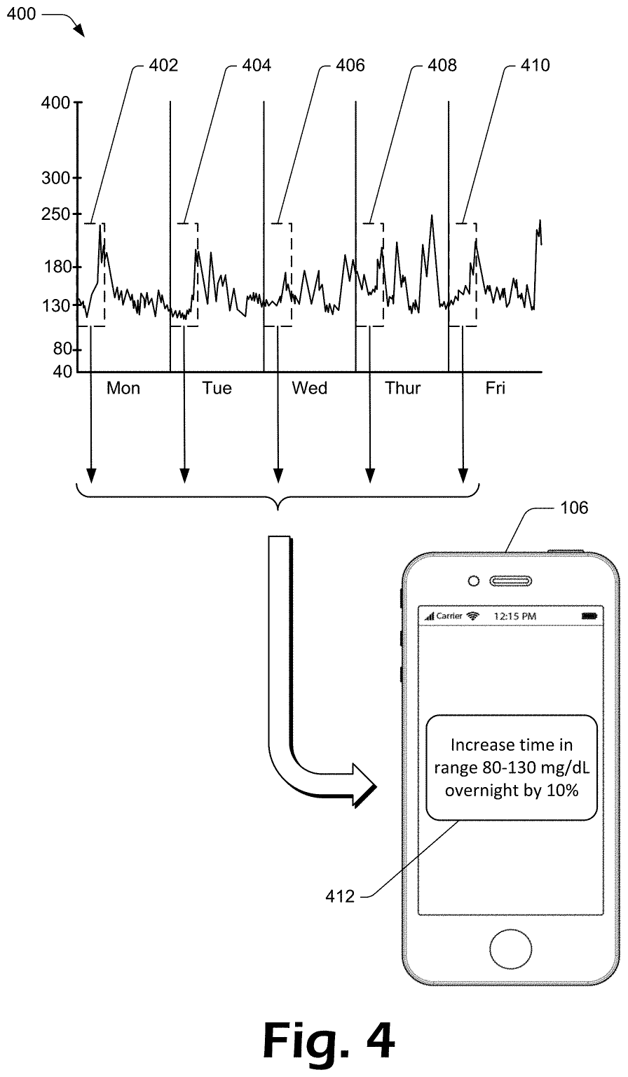

FIG. 4 illustrates an example 400 of providing behavior modification recommendations for improving diabetes management. The example 400 shows a time window of multiple days (illustrated as Monday, Tuesday, Wednesday, Thursday, and Friday) along the horizontal axis and glucose measurements along the vertical axis. Each day has multiple time periods (e.g., night, breakfast, lunch, and dinner) and the glucose measurements during the night time periods in each of the days are illustrated as 402, 404, 406, 408, and 410. A time in range feature 322 is generated for the corresponding time periods (e.g., the night time periods) with a range of 80-130 mg/dL. In the illustrated example 400, the time in range feature 322 is approximately 0.37 (37% of the night time periods are in range). As discussed in more detail below, a pattern is detected given the time in range feature 322 for the night time periods, resulting in behavior modification feedback 412 being displayed on the computing device 106.

Returning to FIG. 3, the pattern detection module 306 receives the different features 322 (e.g., from feature store 326 or directly from feature determination module 304) and detects, from the features 322, patterns in corresponding time periods of a time window. These patterns are patterns that indicate poor (or non-optimal) diabetes management by the user. The pattern detection module 306 can use any of a variety of rules, criteria, or other techniques to identify these patterns.

The pattern detection module 306 identifies patterns based on the features 322 from corresponding time periods in the time window (e.g., patterns in the night time period, patterns in the breakfast time period, patterns in the lunch time period, patterns in the dinner time period, and so forth). The pattern detection module 306 can identify the same or different patterns in the different corresponding time periods. E.g., a pattern may be detected for the night time period and the lunch time period given the time in range feature 322 for those time periods, but no such pattern may be detected for the breakfast and dinner time periods.

In one or more implementations, the pattern detection module 306 uses rules based on target criteria for features 322 that indicate desired values for the features 322. Table I illustrates examples of features 322 and their corresponding target criteria.

TABLE I

| Feature | Criteria |
|---|---|
| Mean | The mean for the glucose measurements in the |
| | corresponding time periods is less than 155 mg/dL |
| Time in | The glucose measurements in the corresponding time |
| range | periods (other than night sleep time periods) are in |
| (not night) | the range of 70-180 mg/dL greater than 70% of the time |
| Time in | The glucose measurements in the night or sleep time |
| range | periods are in the range of 80-130 mg/dL greater than |
| (night) | 70% of the time |
| Time above | The glucose measurements in the corresponding time |
| 180 | periods are above 180 mg/dL less than 25% of the time |
| Time above | The glucose measurements in the corresponding time |
| 250 | periods are above 250 mg/dL less than 5% of the time |
| Time below | The glucose measurements in the corresponding time |
| 70 | periods are below 70 mg/dL less than 1% of the time |
| Max | The maximum glucose measurement in the corresponding |
| glucose | time periods is less than 180 |
| Coefficient | The coefficient of variation for the glucose measurements |
| of variation | in the corresponding time periods is less than 36% |
| Fasting | The fasting glucose is in the range of 80-130 mg/dL |
| glucose | |
| Bedtime | The bedtime glucose is in the range of 80-180 mg/dL |
| glucose | |

The pattern detection module 306 detects, as a pattern that indicates poor (or non-optimal) diabetes management by the user, each feature that does not satisfy its criteria. For example, if the mean for glucose measurements in the corresponding time periods (e.g., the time periods corresponding to after breakfast) is not less than 155 mg/dL, then the pattern detection module 306 detects the glucose measurements for the mean feature in the after breakfast time period as a pattern that indicates poor diabetes management. By way of another example, if the glucose measurements in the corresponding time periods (e.g., the time periods corresponding to after lunch) are in the range of 70-180 mg/dL greater than 70% of the time, then the pattern detection module 306 does not detect the time in range (not night) feature in the after lunch time period as a pattern that indicates poor diabetes management.

The pattern detection module 306 outputs the detected patterns (the features 322 that did not satisfy their criteria) during the time window (e.g., all of the detected patterns for the various features 322 in the various corresponding time periods in the time window) as detected patterns 328. Each detected pattern 328 includes an indication of the detected pattern (e.g., the feature for which the pattern was detected and the corresponding time periods in which the pattern was detected). In one or more implementations, each detected pattern 328 also includes an indication of the feature for which the pattern was detected. For example, if a pattern was detected for the time periods corresponding to after lunch not being in the range of 70-180 mg/dL greater than 70% of the time, the detected pattern 328 includes the amount of time that the glucose measurements were in the range of 70-180 mg/dL for the time periods corresponding to after lunch (e.g., 45%).

In one or more implementations, the detected patterns 328 (or at least the features for which the patterns were detected) are provided to a normalization module 308, which adjusts the features for the detected patterns 328 to a common scale or common units (e.g., a value ranging between 0 and 100, or between 0 and 1). The normalization module 308 outputs the normalized features as normalized features 330. This normalization can be performed using any of a variety of public or proprietary techniques. It should be noted that the normalization module 308 is optional and that normalization need not be performed in certain situations. For example, in some situations if only features having a common scale or common units are used by the pattern detection module 306 (e.g., the time above 180 and the time above 250 features) then there is no need to adjust the features for the detected patterns 328 to a common scale or units.

In one or more embodiments, the normalization performed by normalization module 308 indicates a size of the pattern, and an indication of this size is included in the normalized features 330. The size of the pattern indicates how poorly the criteria for the feature was satisfied. For example, if for a time in range feature, if the time in the particular range (e.g., 70-180 mg/dL) is 45% but the criteria is to be at least 70%, then the size of this pattern can be calculated as

$$100 * ( 1 - \frac{45}{70} )$$

for a size of 35.7, whereas if the time in a different range (e.g., 80-10 mg/dL) is 68% but the criteria is to be at least 70%, then the size of this pattern can be calculated as

$$100 * ( 1 - \frac{68}{70} )$$

for a size of 2. These sizes allow the behavior modification selection module 312 to select behavior modifications based on which pattern has the largest size (e.g., is considered worse or corresponds to the poorer diabetes management behavior).

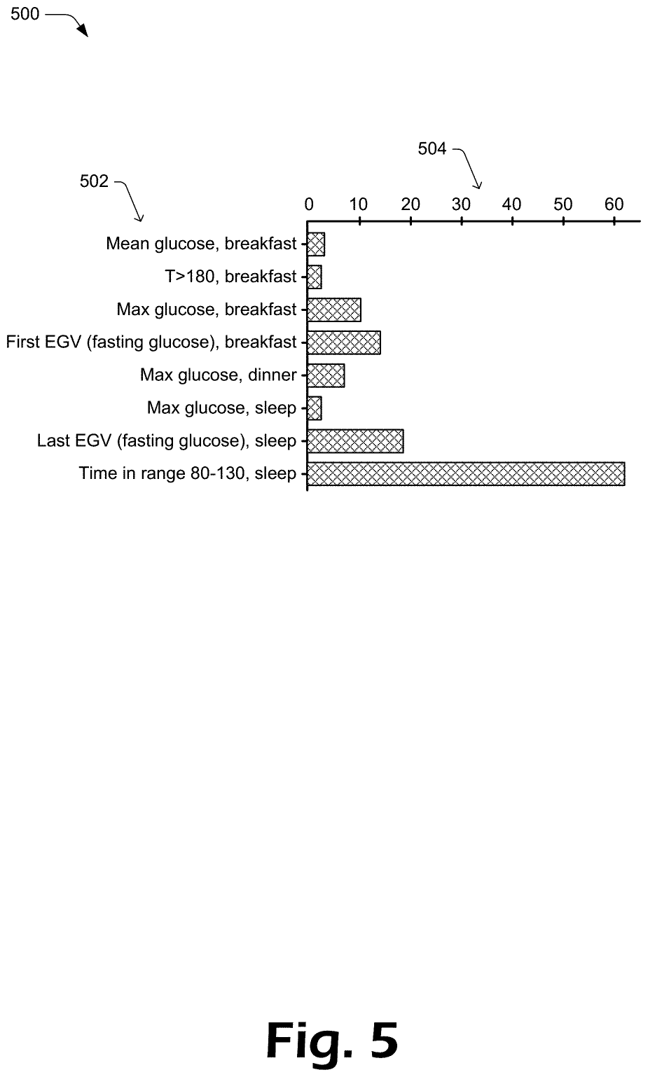

FIG. 5 illustrates an example 500 of sizes of normalized sizes for different detected patterns. The detected patterns (and the time period in which they are detected) 502 are illustrated along the vertical axis, and the sizes 504 are illustrated along the horizontal axis. As illustrated, the detected pattern for the time in range 80-130 mg/dL during the sleep time period has the largest size, which may lead to the behavior modification selection module 312 selecting behavior modification feedback that the time in range 80-130 mg/dL during the sleep time period maps to.

Returning to FIG. 3, in one or more implementations the various patterns that can be detected by the pattern detection module 306 correspond to (are mapped to) one or more topics. The mapping module 310 receives the detected patterns 328 (and optionally the normalized features 330) and maps the detected patterns 328 to one or more topics 332. The topics 332 are also referred to as mapping to one or more patterns. Various behavior modification feedback are grouped into multiple different topics, also referred to as categories. Each such topic includes one or more patterns that are mapped to one or more behavior modification feedback. The mapping module 310 maps the detected patterns 328 to one or more topics 332, and the behavior modification selection module 312 selects behavior modification feedback (from the behavior library 122) corresponding to those one or more topics 332 to provide to the UI module 314 for output as discussed in more detail below. Which detected patterns map to which topic or topics can be specified in various manners, such as by a developer or designer of the behavior modification identification system 120, by a health care provider or professional, and so forth.

The mapping module 310 can map the detected patterns 328 to any of a variety of different topics. For example, one topic of behavior modifications is engagement with a glucose monitoring application, such as glucose monitoring application 116. Patterns that can be mapped to this topic include low engagement with the glucose monitoring application as measured by, e.g., a low number (e.g., less than a threshold number, such as a fixed number (e.g., 3) or a variable number (e.g., less than 2 per hour)) of screen views or launches of the application, no screen views before or after meals, and so forth. In one or more implementations, patterns detected in any of the time periods can be mapped to the engagement with a glucose monitoring application topic. The engagement with a glucose monitoring application topic can be mapped to behavior modification feedback of: 1) check your glucose X number of times per day, 2) check your glucose every day at specified times (e.g., before/after meals, at bedtime, in the morning), 3) set an alarm to remind you to check your glucose, and so forth.

By way of another example, one topic of behavior modifications is post-prandial glucose. Patterns that can be mapped to this topic include high post-prandial glucose peak (e.g., greater than a threshold value, such as a fixed value (e.g., 300 mg/dL) or a variable number (e.g., the highest value the user has had during the time period over a duration of time, such as 2 weeks)), high post-prandial area under the curve (AUC) (e.g., greater than a threshold value, such as a fixed value (e.g., 300 mg/dL) or a variable number (e.g., the highest value the user has had during the time period over a duration of time, such as 2 weeks)), high post-prandial time with glucose levels greater than 250 mg/dl (e.g., greater than a threshold amount of time, such as a fixed amount of time (e.g., 30 minutes) or a variable amount of time (e.g., 10% of the time period)), high post-prandial time with glucose levels greater than 180 mg/dl (e.g., greater than a threshold amount of time, such as a fixed amount of time (e.g., 90 minutes) or a variable amount of time (e.g., 20% of the time period)), high average or mean glucose (e.g., greater than a threshold value, such as a fixed value (e.g., 180 mg/dL) or a variable number (e.g., the average or mean value the user has had during the time period over a duration of time, such as 2 weeks)), low time in a range such as 70-180 mg/dL (e.g., less than a threshold amount of time, such as a fixed amount of time (e.g., 90 minutes) or a variable amount of time (e.g., 20% of the time period)), and so forth. In one or more implementations, patterns detected in any of the time periods can be mapped to the post-prandial glucose topic.

The high post-prandial glucose peak topic can be mapped to behavior modification feedback of: 1) try to keep your post-prandial glucose lower than X by eating food that helps keep your glucose in range (e.g., low carb), 2) annotate what caused elevated (higher than X) post-prandial glucose levels (e.g., type of food, behavior), 3) try to be active after meals to help keep your glucose in range, e.g., for X days next week (or for X days in a row), be active (e.g., try adding a 15 min walk) after meals (e.g., breakfast/lunch/dinner) to control glucose and reduce spikes, and so forth.

By way of another example, one topic of behavior modifications is A1C—GMI (glucose management indicator) or simply GMI. Patterns that can be mapped to this topic include high average or mean glucose (e.g., greater than a threshold value, such as a fixed value (e.g., 180 mg/dL) or a variable number (e.g., the average or mean value the user has had during the time period over a duration of time, such as 2 weeks)). In one or more implementations, patterns detected in the after breakfast time period, after lunch time period, and after dinner time period can be mapped to the A1C—GMI topic.

The A1C—GMI topic can be mapped to behavior modification feedback of: 1) lower average glucose by X, 2) remember to take your medications as prescribed, talk to your doctor, 3) annotate emotions/stress when occurring, 4) try to be more active during the day (e.g., physical activity goal such as aim at completing X steps next week, aim at exercising for X hours next week, perform physical activity X times next week (e.g., walking, cycling, dancing, climbing stairs, jogging, etc.), and so forth.

By way of another example, one topic of behavior modifications is overnight glucose. Patterns that can be mapped to this topic include high average or mean nocturnal glucose (e.g., greater than a threshold value, such as a fixed value (e.g., 180 mg/dL) or a variable number (e.g., the highest value the user has had during the time period over a duration of time, such as 2 weeks)), low nocturnal time in a range such as 70-180 mg/dL (e.g., less than a threshold amount of time, such as a fixed amount of time (e.g., 30 minutes) or a variable amount of time (e.g., 10% of the time period)), low nocturnal time in a range such as 80-130 mg/dL (e.g., less than a threshold amount of time, such as a fixed amount of time (e.g., 15 minutes) or a variable amount of time (e.g., 5% of the time period)), high nocturnal time in hyperglycemic range (e.g., greater than a threshold amount of time, such as a fixed amount of time (e.g., 30 minutes) or a variable amount of time (e.g., 10% of the time period)), high bedtime glucose (e.g., greater than a threshold value, such as a fixed value (e.g., 250 mg/dL) or a variable number (e.g., the highest value the user has had during the time period over a duration of time, such as 2 weeks)), low bedtime glucose (e.g., less than a threshold value, such as a fixed value (e.g., 70 mg/dL) or a variable number (e.g., the lowest value the user has had during the time period over a duration of time, such as 2 weeks)), high nocturnal time with glucose levels greater than 250 mg/dl (e.g., greater than a threshold amount of time, such as a fixed amount of time (e.g., 30 minutes) or a variable amount of time (e.g., 10% of the time period)), high nocturnal time with glucose levels greater than 180 mg/dl (e.g., greater than a threshold amount of time, such as a fixed amount of time (e.g., 90 minutes) or a variable amount of time (e.g., 20% of the time period)), and so forth. In one or more implementations, patterns detected in the after sleep time period can be mapped to the overnight glucose topic.

The overnight glucose topic can be mapped to behavior modification feedback of: 1) increase your overnight time in range by a X %, 2) remember to take your medications as prescribed, talk to your doctor, 3) try to eat a dinner that won't raise your glucose too high (e.g., smaller portions, fewer carbs), 4) try not to eat close to bedtime (e.g., try not to eat after X PM, set an alarm as a reminder), 5) check your glucose before going to bed to see if you are in range (self-reflection), and so forth.

By way of another example, one topic of behavior modifications is glucose variability. Patterns that can be mapped to this topic include high values for high variability metrics (e.g., less than a threshold number, such as a fixed number (e.g., 2) or a variable number (e.g., the highest value the user has had during the time period over a duration of time, such as 2 weeks)), such as coefficient of variation or time spent in |ROC|>2, and so forth. In one or more implementations, patterns detected in any of the time periods can be mapped to the glucose variability topic.

The glucose variability topic can be mapped to behavior modification feedback of: 1) for X days next week, choose low carbs foods and limit high carb foods, 2) for X days next week, pay attention to how often you have meal-related glucose spikes, 3) try to eat no more than X times during the day, 4) check your glucose before/after a meal to see if you are in range and to understand how specific food impacts your glucose (self-reflection), 5) check and annotate carbs content on foods you eat more often (self-reflection), 6) annotate emotions/stress when occurring next week, and so forth.

By way of another example, one topic of behavior modifications is fasting glucose. Patterns that can be mapped to this topic include high estimated fasting glucose (e.g., greater than a threshold value, such as a fixed value (e.g., 250 mg/dL) or a variable number (e.g., the highest value the user has had during the time period over a duration of time, such as 2 weeks)). In one or more implementations, patterns detected at the beginning of the after breakfast time period and the ending of the sleep time period can be mapped to the fasting glucose topic. The fasting glucose topic can be mapped to behavior modification feedback of: 1) try to eat a dinner that won't raise your glucose too high (smaller portions, fewer carbs), 2) pay attention to how many hours you leave between your last and first meals, 3) try to leave X hours between dinner and breakfast, and so forth.

By way of another example, one topic of behavior modifications is hyperglycemia (also referred to as sustained hyperglycemia). Patterns that can be mapped to this topic include high time greater than 180 mg/dl (e.g., greater than a threshold amount of time, such as a fixed amount of time (e.g., 30 minutes) or a variable amount of time (e.g., 10% of the time period)), high time greater than 250 mg/dl (e.g., greater than a threshold amount of time, such as a fixed amount of time (e.g., 10 minutes) or a variable amount of time (e.g., 3% of the time period)), and so forth. In one or more implementations, patterns detected in the after breakfast time period, after lunch time period, and after dinner time period can be mapped to the hyperglycemia topic.

The hyperglycemia topic can be mapped to behavior modification feedback of: 1) if high time is greater than 15% talk to your doctor, 2) remember to take your medications as prescribed, 3) annotate emotions/stress when occurring next week, 4) try to be more active during the day (physical activity), e.g., aim at completing X steps next week, aim at exercising for X hours next week, perform physical activity X times next week (e.g., walking, cycling, dancing, climbing stairs, jogging, etc.), and so forth.

By way of another example, one topic of behavior modifications is time in range. Patterns that can be mapped to this topic include low time in a range such as 70-180 mg/dL (e.g., less than a threshold amount of time, such as a fixed amount of time (e.g., 90 minutes) or a variable amount of time (e.g., 20% of the time period)). In one or more implementations, patterns detected in the after breakfast time period, after lunch time period, and after dinner time period can be mapped to the time in range topic. The time in range topic can be mapped to behavior modification feedback of: increase time in range by X, and so forth.

By way of another example, one topic of behavior modifications is hypoglycemia. Patterns that can be mapped to this topic include high time (e.g., greater than a threshold amount of time, such as a fixed amount of time (e.g., 30 minutes) or a variable amount of time (e.g., 10% of the time period)) in a hypoglycemic range (e.g., less than 70 mg/dL). In one or more implementations, patterns detected in any of the time periods can be mapped to the hypoglycemia topic. The hypoglycemia topic can be mapped to behavior modification feedback of: 1) talk to your doctor, 2) consider these suggestions (education content that could be added in the message to the user), such as do you know the rule of 15 when you're less than 70, check your glucose before you are physically active, check your glucose before you drive, and so forth.

In one or more implementations, patterns detected in any of the time periods can be mapped to a topic. Additionally or alternatively, patterns detected in only certain time periods may be mapped to a topic. For example, patterns mapped to the fasting glucose topic may be detected at the end of the sleep time period or the beginning of the after breakfast time period, but not during other time periods. By way of another example, patterns mapped to the overnight glucose topic may be detected during the sleep time period but not during other time periods.

In one or more implementations, some patterns have a one-to-one mapping to topics. For example, the high estimated fasting glucose pattern is mapped to just the fasting glucose topic. However, other patterns may potentially map to multiple topics. For example, the high time greater than 180 mg/dl pattern may be mapped to the post-prandial glucose topic or the hyperglycemia topic. For such patterns, the mapping module 310 determines which topic to map the pattern to based on how many time periods the pattern is identified in.

For example, if one or both of the high post-prandial time with glucose levels greater than 180 mg/dl or high post-prandial time with glucose levels greater than 250 mg/dl patterns are detected in less than a threshold number of time periods in a day or other 24-hour period (e.g., 3 time periods), then the pattern is mapped to the post-prandial glucose topic. However, if the patterns are detected in at least the threshold number of time periods in a day or 24-hour period (e.g., at least 3 time periods), then the pattern is mapped to the hyperglycemia topic.

By way of another example, if the high average or mean glucose pattern is detected in less than a threshold number of time periods in a day or other 24-hour period (e.g., 3 time periods), then the pattern is mapped to the post-prandial glucose topic. However, if the pattern is detected in at least the threshold number of time periods in a day or 24-hour period (e.g., at least 3 time periods), then the pattern is mapped to the GMI topic.

By way of another example, if the low time in a range such as 70-180 mg/dL pattern is detected in less than a threshold number of time periods in a day or other 24-hour period (e.g., 3 time periods), then the pattern is mapped to the post-prandial glucose topic. However, if the pattern is detected in at least the threshold number of time periods in a day or 24-hour period (e.g., at least 3 time periods), then the pattern is mapped to the time in range topic.

The mapping module 310 maps multiple patterns to the same topic to reduce redundancy in situations in which the same behavior modification feedback could be provided to improve diabetes management. For example, the same behavior modification feedback could be provided to improve diabetes management in situations in which a pattern of high post-prandial glucose peak after lunch and a pattern of high post-prandial time with glucose levels greater than 250 mg/dl after lunch are detected. By mapping both of these patterns to the “post-prandial glucose” topic, the behavior modification identification system 120 can avoid providing the same behavior modification feedback if both patterns are detected in a time period.

Various different example times, glucose levels, and other values are discussed with reference to the detected patterns 328. It should be noted that these various different times, glucose levels, and other values are just examples and that various other times, glucose levels, and other values can be used instead.

The mapping module 310 outputs one or more topics 332 to the behavior modification selection module 312. The one or more topics 332 include each topic that a detected pattern 328 is mapped to. In situations in which multiple patterns map to the same topic, the one or more topics 332 need include (and typically does include) that topic only once. However, the one or more topics 332 may include the same topic for different time periods, such as in situations in which a pattern mapped to the same topic in multiple different time periods. In one or more implementations, for each topic 332, the mapping module 310 also provides one or both of the detected patterns 328 that mapped to the topic 332 and the normalized features 330.

The various topics to which patterns are mapped correspond to (are mapped to) one or more behavior modification feedback. The behavior modification selection module 312 receives the one or more topics 332 (and optionally the normalized features 330) and selects behavior modification feedback from the behavior library 122 to provide to the UI module 314 for output. In one or more embodiments, the behavior modification selection module 312 maps each topic 332 to particular behavior modification feedback (e.g., a particular message or text). Each of the behavior modification feedback in the behavior library 122 is also referred to as being mapped to a topic 332. The mappings between topics 332 and behavior modification feedback can be specified in various manners, such as by a developer or designer of the behavior modification identification system 120, by a health care provider or professional, and so forth.

The behavior modification feedback in the behavior library 122 can be obtained from any of a variety of sources. For example, the behavior modification feedback can be obtained from health care providers or professionals, a clinician, standard of care or other publications, and so forth. In one or more implementations, the behavior library 122 includes user input or specified behavior modification feedback, allowing the user to select or create behavior modification feedback that they would like to see if the pattern that maps to their behavior modification feedback is detected. The behavior modification feedback also optionally includes additional educational material or links to resources (e.g., via the Internet) for additional information describing the behavior modification feedback, describing terms in the behavior modification feedback, and so forth. E.g., if a behavior modification feedback is to try to eat a dinner with fewer carbs, the behavior modification feedback can include links to guides identifying foods or recipes that are low carb.

In one or more implementations, the behavior modification selection module 312 selects all behavior modification feedback that is mapped to by at least one topic 332 to provide to UI module 314.

Additionally or alternatively, in situations in which multiple behavior modification feedback is mapped to by different topics, behavior modification selection module 312 selects one or more of the mapped to behavior modification feedback to provide to UI module 314. The behavior modification selection module 312 can select one or more of the mapped to behavior modification feedback in various manners, such as randomly or pseudorandomly selecting one of the mapped to mapped to behavior modification feedback. Additionally or alternatively, the behavior modification selection module 312 can prioritize the multiple mapped to behavior modification feedback and select one or more of the multiple mapped to behavior modification feedback a highest priority (or priorities). For example, the mapped to behavior modification feedback having the highest priority is selected.

The behavior modification selection module 312 optionally uses various criteria to determine which of the multiple mapped to behavior modification feedback to select. These criteria can be based on various factors, such as how recently the pattern that mapped to a topic was detected, a ranking or prioritization of behavior modification feedback, topics, or categories of behavior modification feedback, and so forth. For example, the patterns corresponding to the normalized features 330 have various sizes as discussed above. Accordingly, the behavior modification feedback mapped to by a topic to which the pattern having the largest size is mapped is selected.

By way of another example, behavior modification feedback mapped to by a topic to which a pattern that was detected less recently is mapped is selected over behavior modification feedback mapped to by a topic to which a pattern that was detected more recently is mapped. E.g., this allows behavior modification feedback mapped to by different topics to be selected as behavior modification feedback 334 and avoids repeating behavior modification feedback too frequently.

By way of another example, behavior modification feedback corresponding to certain topics or categories can be selected over behavior modification feedback corresponding to other topics or categories. For example, behavior modification feedback mapped to by a hypoglycemia topic may be selected over behavior modification feedback mapped to by an engagement with a glucose monitoring application topic. E.g., this allows behavior modification feedback mapped to by topics or categories deemed more important to the user's health to be selected before behavior modification feedback mapped to by topics or categories deemed less important.

By way of another example, behavior modification feedback designated (e.g., by a developer or designer of the behavior modification selection module 312) to be more urgent or safety-related is selected over behavior modification feedback that is less urgent or safety-related. E.g., this allows behavior modification feedback corresponding to urgent or safety-related features (e.g., not staying within ranges or exceeding threshold glucose levels) to be selected over other non-urgent or non-safety-related behavior modification feedback and display or otherwise present more critical behavior modification feedback to the user.

By way of another example, behavior modification feedback designated as being higher priority (e.g., by the user 102) is selected over behavior modification feedback that is designated as being lower priority (e.g., by the user 102). E.g., this allows behavior modification feedback that is of greater interest to the user to be displayed or otherwise presented rather than behavior modification feedback that is of less interest to the user.

By way of another example, behavior modification feedback designated as being helpful by the user 102 or associated with an improvement in diabetes management is selected over behavior modification feedback that is not designated as being helpful by the user 102 or did not lead to an improvement in diabetes management. E.g., this allows behavior modification feedback that is more helpful to the user, or that previously resulted in an improvement in diabetes management, to be presented to the user again (optionally customized with updated values, such as walk 4 times per week rather than 2 times per week) rather than other behavior modification feedback.

Furthermore, the behavior modification selection module 312 can receive additional data 324, which can be any additional data that may be used to identify poor diabetes management as discussed above. The additional data 324 may include data from various sources, for example applications or programs of the computing device 106, user input by the user 102, input by a healthcare provider (e.g., the user's doctor or nurse), external devices such as activity trackers, and so forth.

The additional data 324 can include data that relates to user interactions with the computing device 106, with the display of the computing device 106, or with other system components that indicate level of engagement with diabetes management as discussed above.

By way of another example, additional data 324 can include activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), and so forth. Activity data can be received from various sources, such as wearable glucose monitoring device 104, an activity tracking application running on computing device 106, an activity or fitness tracker worn by the user 102, and so forth.

By way of another example, additional data 324 can include data regarding sleeping patterns of the user. E.g., additional data 324 can include data indicating times when the user is sleeping, the sleep state (e.g., Stage 1, Stage 2, Stage 3, or rapid eye movement (REM) sleep) of the user at particular times, and so forth.

By way of another example, additional data 324 can include data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

By way of another example, additional data 324 can include meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

By way of another example, additional data 324 can include sleep data, such as data indicating minutes of the day when the user was sleeping. Sleep data can be received from various sources, such as wearable glucose monitoring device 104, a sleep tracking application running on computing device 106, an activity or fitness tracker worn by the user 102, and so forth.

By way of another example, additional data 324 can include medication data. E.g., this medication data can include timestamps of when user 102 took medicine (e.g., basal insulin) and what medicine was taken (which can be used to determine whether the user 102 is taking his or her medicine at the prescribed times or intervals), indications of changes in medicines (e.g., changes in types or dosages of medicines taken), and so forth.

By way of another example, additional data 324 can include data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

By way of another example, additional data 324 can include current health data. E.g., this current health data can include whether a user is currently sick (e.g., has a cold, has a virus), whether a user is currently recovering from an operation or other procedure, diseases or chronic conditions that the user is currently diagnosed with (e.g., kidney disease or liver disease), and so forth.

In one or more implementations, the behavior modification selection module 312 can select one or more of the mapped to behavior modification feedback based on the additional data 324, such as by using the additional data 324 to prioritize behavior modification feedback or filter out behavior modification feedback. For example, the behavior modification selection module 312 would filter out (not select) behavior modification feedback to perform physical activity X times next week if the additional data 324 indicates the user is sick or recovering from foot surgery. By way of another example, the behavior modification selection module 312 would filter out (not select) behavior modification feedback to try to be active after meals to help keep your glucose in range if the additional data 324 indicates the user is regularly active after meals. By way of another example, the behavior modification selection module 312 could select or give a higher priority to behavior modification feedback to try to be active after meals to help keep your glucose in range if the additional data 324 indicates the user is rarely (or never) active after meals.

In one or more implementations, the behavior modification selection module 312 communicates with a behavior modification feedback customization module 336. Some behavior modification feedback includes variables or blanks that are altered based on the particular user 102. The behavior modification feedback customization module 336 receives one or more of the glucose measurements 114, the grouped measurements 320, the features 322 and the additional data 324, and alters or fills in these variables or blanks in the behavior modification feedback to customize the glucose measurement feedback to the user 102. For example, various different behavior modification feedback discussed above include X, such as check your glucose X number of times per day or try to keep your post-prandial glucose lower than X by eating food that helps keep your glucose in range (e.g., low carb). The behavior modification feedback customization module 336 determines a value (e.g., a specific number or range of numbers) to replace the X with so that the behavior modification feedback 334 displayed to the user is “keep your post-prandial glucose lower than 197 by eating food that helps keep your glucose in range (e.g., low carb)” rather than simply “keep your post-prandial glucose lower by eating food that helps keep your glucose in range (e.g., low carb)” or replacing the X with a standard value (e.g., 180).

The behavior modification feedback customization module 336 customizes behavior modification feedback customization module 336 in various manners. In one or more implementations, the behavior modification feedback customization module 336 adds a default value (e.g., 50) to a glucose measurement 114 or a feature 322. For example, a feature 322 may be the mean glucose measurement 114 at the beginning of corresponding time periods (e.g., dinner time periods). The behavior modification feedback customization module 336 adds the default value (e.g., 50) to the mean value (e.g., 147), resulting in the customized behavior modification feedback of “keep your post-prandial glucose lower than 197 by eating food that helps keep your glucose in range (e.g., low carb).”

Additionally or alternatively, the behavior modification feedback customization module 336 analyzes the various data it receives to determine a realistic, actionable goal for the user 102. For example, if the user does not regularly walk after meals, the behavior modification feedback customization module 336 can determine to customize behavior modification feedback to suggest walking two times per week after meals. However, if the user regularly walks two times per week after meals, the behavior modification feedback customization module 336 can determine to customize behavior modification feedback to suggest walking four times per week after meals. By way of another example, if the user does not check their glucose level via glucose monitoring application 116 each day, the behavior modification feedback customization module 336 can determine to customize behavior modification feedback to suggest “check your glucose 3 times per day”. However, if the user regularly checks their glucose level via glucose monitoring application 116 two times each day, the behavior modification feedback customization module 336 can determine to customize behavior modification feedback to suggest “check your glucose 6 times per day”.

The UI module 314 receives the selected behavior modification feedback 334 and causes the behavior modification feedback 334 to be displayed or otherwise presented (e.g., at computing device 106). This display or other presentation can take various forms, such as a static text display, graphic or video display, audio presentation, combinations thereof, and so forth. In one or more implementations, different topics or categories of behavior modification feedback are displayed or otherwise presented in different manners. For example, behavior modification feedback corresponding to different topics or categories can be displayed using different colors, different icons, and so forth. The example 400 of FIG. 4 illustrates an example of behavior modification feedback as behavior modification feedback 412.

The behavior modification identification system 120 generates and displays or otherwise communicates the selected behavior modification feedback 334 at various intervals. In one or more embodiments, the behavior modification feedback 334 is generated and displayed or otherwise communicated weekly, such as Sunday evening so that the behavior modification feedback 334 is available to the user at the beginning of the week (e.g., giving the user a goal to achieve for the week). Additionally or alternatively, other timings can be used, such as bi-weekly, daily, bi-daily, and so forth. Additionally or alternatively, the behavior modification selection module 312 may display or otherwise communicate high priority behavior modification feedback 334 immediately, such as in situations where there is an immediate safety risk (e.g., due to hypoglycemia).

In one or more implementations, the behavior modification selection module 312 tracks the behavior modification feedback 334 provided to the UI module 314, determines whether the behavior modification feedback 334 was followed, and provides additional behavior modification feedback 334 based on whether the behavior modification feedback 334 was followed. For example, if the behavior modification feedback 334 is to complete 35,000 steps next week, the additional data 324 can include activity data indicating whether the user completed 35,000 steps over the week. E.g., behavior modification feedback congratulating the user on successfully following the previous week's behavior modification feedback may be provided if the user completed 35,000 steps, or behavior modification feedback encouraging the user to keep up the good work if they did not complete 35,000 steps but came close or had significant improvement over previous weeks.

The behavior modification identification system 120 optionally takes additional actions based on the behavior modification feedback 334. In one or more implementations, these actions include notifying the glucose monitoring application 116 or the wearable glucose monitoring device 104 that the frequency with which glucose measurements 114 are produced can be reduced. For example, if the behavior modification identification system 120 identifies that no patterns are detected for particular time periods (e.g., corresponding to sleep), the behavior modification identification system 120 notifies the glucose monitoring application 116 or wearable glucose monitoring device 104 that the frequency with which glucose measurements 114 are produced can be reduced (e.g., from every 5 minutes to every 10 minutes), reducing the power expended to produce glucose measurements 114.

Additionally or alternatively, these actions include determining whether to recommend ongoing CGM use (e.g., starting a new sensor immediately after the current sensor expires) or whether it may be appropriate to take a break from using CGM and starting a new sensor at some later date. For example, if the behavior modification identification system 120 identifies that patterns are detected regularly in all time periods, the behavior modification identification system 120 recommends (e.g., via display or other presentation to the user) ongoing CGM use.

Discussions are also included herein with reference to behavior modification feedback being displayed or otherwise presented to the user 102. Additionally or alternatively, the behavior modification feedback is communicated to or otherwise delivered to others, such as a clinician (e.g., the user's primary care physician or nurse), a pharmacist, and so forth. This can serve to partially automate some of the manual effort of reviewing raw glucose or other diabetes management data that a clinician may have to do on their own in the absence of generated behavior modification feedback. Additionally or alternatively, rather than providing the behavior modification feedback 334, the behavior modification selection module 312 can provide the features 322, normalized features 330, or detected patterns 328 may be provided to the clinician, pharmacist, or others, enabling them to apply their own preferred behavior modification selection (if any) in determining which behavior modification feedback should be passed along to the user 102.

Discussions are also included herein with reference to determining particular time periods within the time window. These time periods can be determined prior to analysis of the features 322 by the pattern detection module 306 to detect patterns in corresponding time periods of the time window. Additionally or alternatively, these time periods may be determined at a later time. In one or more implementations, the pattern detection module 306 or another module may analyze the features 322 in various time ranges within the day (e.g., 30-minute, 60-minute, 120-minute, etc. ranges of time at some interval such as 5 or 10 minutes). If the pattern detection module 306 detects a pattern in one of those time ranges on a single day, that time range is treated by the behavior modification identification system 120 as a time period. The time range is optionally expanded (e.g., by 10 minutes on either side) to create the time period. The corresponding time periods in other time windows (e.g., the same time range in other days) are then used to determine whether there is a pattern in the corresponding time periods across multiple time windows.

For example, assume the time window is one day. The pattern detection module 306 may begin analyzing the features 322 over the previous 60 minutes beginning at 1:00 am on a particular day, moving forward in 10 minute intervals. When analyzing the features 322 for the time range of 1:20 am-2:20 am, the pattern detection module 306 may detect a pattern in the time range of 1:20 am-2:20 am. The pattern detection module 306 uses the time range of 1:20 am-2:20 am (or expands the time range to 1:10 am-2:30 am) as a time period and analyzes the features 322 for that time period across multiple days (e.g., the previous week) to detect whether there is a pattern in the corresponding time periods of the multiple days.

Additionally or alternatively, in one or more implementations the behavior modification identification system 120 (e.g., the behavior modification selection module 312) maintains a record of one or more of detected patterns 328, features 322, and behavior modification feedback 334. The behavior modification identification system 120 (e.g., the behavior modification selection module 312) analyzes the detected patterns 328 or features 322 over longer ranges of time, such as months or years, and identifies improvements over those longer ranges of time. For example, the behavior modification identification system 120 compares the detected patterns 328 or features 322 for a current 1-week time window to the detected patterns 328 or features 322 of a 1-week time window six months or a year ago. Improvements in diabetes management identified by this comparison (e.g., as indicated by the features 322 or by patterns detected six months or a year ago that are not detected in the current week) can be identified to the user via UI module 314. E.g., a congratulatory message identifying the improvement may be communicated, displayed, or otherwise presented to the user or other person (e.g., health care provider or clinician). The behavior modification feedback that was previously provided to the user (e.g., six months or a year ago) can also be communicated, displayed, or otherwise presented to the user or other person, providing an indication of what behavior modification feedback was followed by the user that resulted in the improvement in diabetes management.

Discussions are also included herein with reference to detecting patterns, mapping patterns to topics, and mapping topics to behavior modification feedback. Additionally or alternatively, the techniques discussed herein need not use topics. In such situations detected patterns can be mapped to behavior modification feedback. Which patterns map to which behavior modification feedback can be specified in various manners, such as by a developer or designer of the behavior modification identification system, by a health care provider or professional, and so forth.

Having discussed exemplary details of the techniques for user interfaces for glucose insight presentation, consider now some examples of procedures to illustrate additional aspects of the techniques.

Example Procedures

This section describes examples of procedures for implementing behavior modification feedback for improving diabetes management. Aspects of the procedures may be implemented in hardware, firmware, or software, or a combination thereof The procedures are shown as a set of blocks that specify operations performed by one or more devices and are not necessarily limited to the orders shown for performing the operations by the respective blocks.

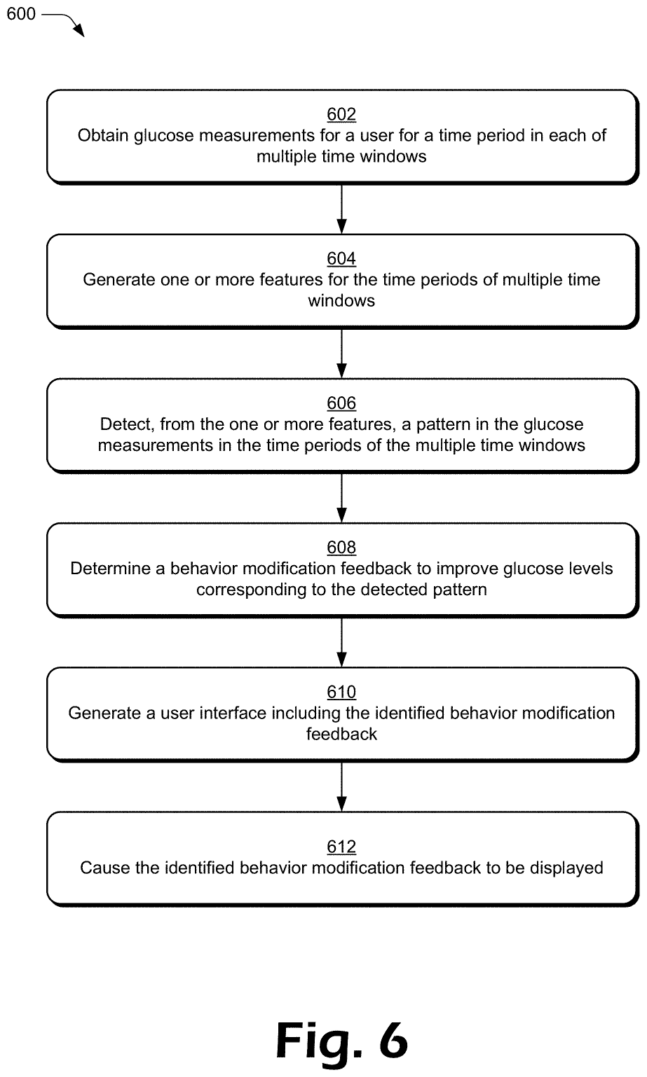

FIG. 6 depicts a procedure 600 in an example of implementing behavior modification feedback for improving diabetes management. Procedure 600 is performed, for example, by a behavior modification identification system, such as the behavior modification identification system 120.

Glucose measurements for a user for a time period in each of multiple time windows are obtained (block 602). The glucose measurements are obtained for corresponding time periods across the multiple time windows, such as for the lunch time periods on multiple days. These glucose measurements are obtained from a glucose sensor of, for example, a continuous glucose level monitoring system with the glucose sensor being inserted at an insertion site of the user.

One or more features for the time periods of the multiple time windows are generated (block 604). These one or more features are generated from the glucose measurements.

A pattern in the glucose measurements in the time periods of the multiple time windows is detected (block 606). This detection is made based on the generated features for the time periods of the multiple time windows.

A behavior modification feedback to improve glucose levels corresponding to the detected pattern is determined (block 608). The detected pattern may be mapped to a topic that is mapped to one or more behavior modification feedback, one or more of which is selected in block 608. Additionally or alternatively, the detected pattern may be mapped to or correspond to multiple behavior modification feedback, and one or more of the multiple behavior modification feedback is selected in block 608.

A user interface including the identified behavior modification feedback is generated (block 610). The identified diabetes management feedback is caused to be displayed (block 612) or otherwise presented. Additionally or alternatively, the identified diabetes management feedback can be communicated to or otherwise presented to a clinician, pharmacist, other health care provider, and so forth.

Example System and Device

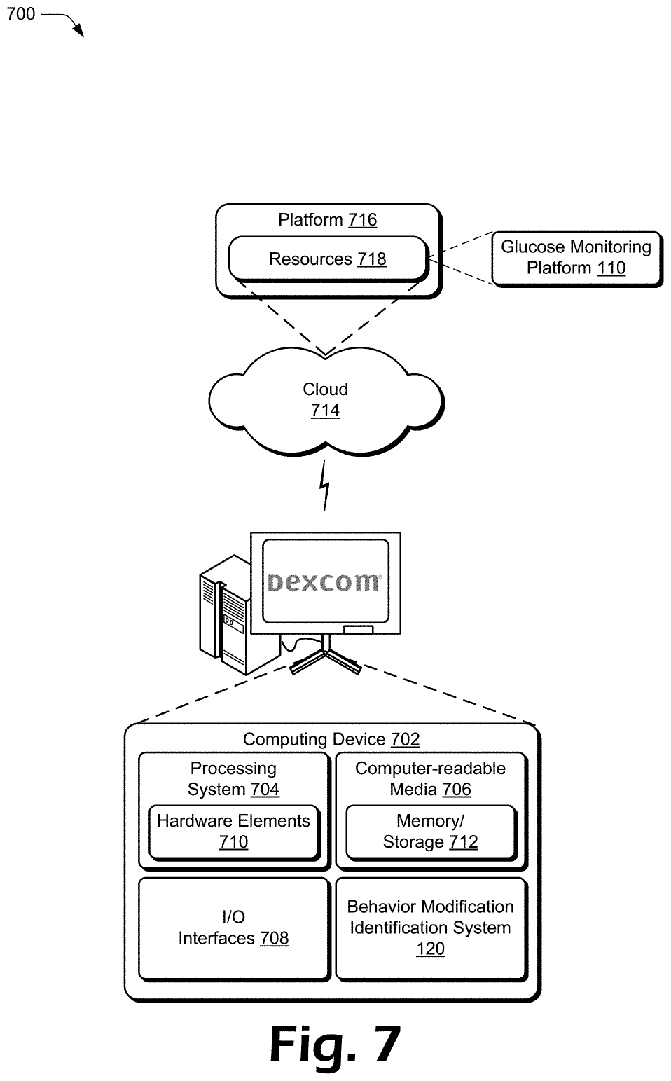

FIG. 7 illustrates an example of a system generally at 700 that includes an example of a computing device 702 that is representative of one or more computing systems and/or devices that may implement the various techniques described herein. This is illustrated through inclusion of the behavior modification identification system 120. The computing device 702 may be, for example, a server of a service provider, a device associated with a client (e.g., a client device), an on-chip system, and/or any other suitable computing device or computing system.

The example computing device 702 as illustrated includes a processing system 704, one or more computer-readable media 706, and one or more I/O interfaces 708 that are communicatively coupled, one to another. Although not shown, the computing device 702 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 704 is representative of functionality to perform one or more operations using hardware. Accordingly, the processing system 704 is illustrated as including hardware elements 710 that may be configured as processors, functional blocks, and so forth. This may include implementation in hardware as an application specific integrated circuit or other logic device formed using one or more semiconductors. The hardware elements 710 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 706 is illustrated as including memory/storage 712. The memory/storage 712 represents memory/storage capacity associated with one or more computer-readable media. The memory/storage component 712 may include volatile media (such as random access memory (RAM)) and/or nonvolatile media (such as read only memory (ROM), Flash memory, optical disks, magnetic disks, and so forth). The memory/storage component 712 may include fixed media (e.g., RAM, ROM, a fixed hard drive, and so on) as well as removable media (e.g., Flash memory, a removable hard drive, an optical disc, and so forth). The computer-readable media 706 may be configured in a variety of other ways as further described below.

Input/output interface(s) 708 are representative of functionality to allow a user to enter commands and information to computing device 702, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 702 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 702. By way of example, computer-readable media may include “computer-readable storage media” and “computer-readable signal media.”

“Computer-readable storage media” may refer to media and/or devices that enable persistent and/or non-transitory storage of information thereon in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 702, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, communication media include wired media such as a wired network or direct-wired connection, and wireless media such as acoustic, RF, infrared, and other wireless media.

As previously described, hardware elements 710 and computer-readable media 706 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 710. The computing device 702 may be configured to implement particular instructions and/or functions corresponding to the software and/or hardware modules. Accordingly, implementation of a module that is executable by the computing device 702 as software may be achieved at least partially in hardware, e.g., through use of computer-readable storage media and/or hardware elements 710 of the processing system 704. The instructions and/or functions may be executable/operable by one or more articles of manufacture (for example, one or more computing devices 702 and/or processing systems 704) to implement techniques, modules, and examples described herein.

The techniques described herein may be supported by various configurations of the computing device 702 and are not limited to the specific examples of the techniques described herein. This functionality may also be implemented all or in part through use of a distributed system, such as over a “cloud” 714 via a platform 716 as described below.

The cloud 714 includes and/or is representative of a platform 716 for resources 718. The platform 716 abstracts underlying functionality of hardware (e.g., servers) and software resources of the cloud 714. The resources 718 may include applications and/or data that can be utilized while computer processing is executed on servers that are remote from the computing device 702. Resources 718 can also include services provided over the Internet and/or through a subscriber network, such as a cellular or Wi-Fi network.

The platform 716 may abstract resources and functions to connect the computing device 702 with other computing devices. The platform 716 may also serve to abstract scaling of resources to provide a corresponding level of scale to encountered demand for the resources 718 that are implemented via the platform 716. Accordingly, in an interconnected device embodiment, implementation of functionality described herein may be distributed throughout the system 700. For example, the functionality may be implemented in part on the computing device 702 as well as via the platform 716 that abstracts the functionality of the cloud 714.

In some aspects, the techniques described herein relate to a method implemented in a continuous glucose level monitoring system, the method including: obtaining, from a glucose sensor of the continuous glucose level monitoring system and for each time window of multiple time windows, glucose measurements measured for a user for a first time period of multiple time periods of the time window, the glucose sensor being inserted at an insertion site of the user; generating, from the glucose measurements, one or more features for the first time periods of the multiple time windows; detecting, from the one or more features for the first time periods of the multiple time windows, a pattern in the glucose measurements in the first time periods of the multiple time windows; determining a behavior modification feedback to improve glucose levels corresponding to the pattern; generating a user interface including the behavior modification feedback; and causing the user interface to be displayed.

In some aspects, the techniques described herein relate to a method, wherein each time window includes one day, the multiple time windows include multiple days, and each of the multiple time periods includes a different multi-hour period of time during a day.

In some aspects, the techniques described herein relate to a method, wherein the multiple time periods include an overnight time period, an after breakfast time period, an after lunch time period, and an after dinner time period.

In some aspects, the techniques described herein relate to a method, further including receiving user input specifying, for each of the multiple time periods, the multi-hour period of time during the day for the time period.

In some aspects, the techniques described herein relate to a method, further including automatically learning, by a machine learning system, at least one of the multi-hour periods of time of the day.

In some aspects, the techniques described herein relate to a method, wherein each time window includes one week, the multiple time windows include multiple weeks, and each of the multiple time periods includes a different day in a week.

In some aspects, the techniques described herein relate to a method, wherein the detecting a pattern includes determining that criteria for a feature of the one or more features is not satisfied.

In some aspects, the techniques described herein relate to a method, wherein the pattern is one of multiple patterns detected in the glucose measurements, each of the multiple patterns being one of the one or more features for which corresponding criteria is not satisfied, the method further including normalizing the multiple patterns to generate a size for each of the multiple patterns.

In some aspects, the techniques described herein relate to a method, wherein the determining the behavior modification feedback includes selecting behavior modification feedback corresponding to one of the multiple patterns having a largest size.

In some aspects, the techniques described herein relate to a method, further including: generating a numeric value for the user based on the glucose measurements or the one or more features; and customizing the behavior modification feedback to the user by including at least one numeric value in the behavior modification feedback.

In some aspects, the techniques described herein relate to a method, wherein the pattern is one of multiple patterns detected in the glucose measurements, and the determining the behavior modification feedback includes selecting behavior modification feedback mapped to by one of multiple topics that is mapped to by one of the multiple patterns.

In some aspects, the techniques described herein relate to a method, wherein the pattern is one of multiple patterns detected in the glucose measurements, and the determining the behavior modification feedback includes selecting behavior modification feedback corresponding to one of the multiple patterns.

In some aspects, the techniques described herein relate to a method, further including receiving activity data for the user from an activity tracker, and wherein the selecting behavior modification feedback includes not selecting behavior modification feedback indicating to perform activity that the activity data indicates the user is already performing

In some aspects, the techniques described herein relate to a method, further including: subsequently determining whether the behavior modification in the behavior modification feedback was performed by the user; and providing additional feedback congratulating the user in response to determining that the behavior modification in the behavior modification feedback was performed by the user.

In some aspects, the techniques described herein relate to a computing device including: a processor; a display device; and computer-readable storage media having stored thereon multiple instructions of an application that, responsive to execution by the processor, cause the processor to: obtain, from a glucose sensor of a continuous glucose level monitoring system and for each time window of multiple time windows, glucose measurements measured for a user for a first time period of multiple time periods of the time window, the glucose sensor being inserted at an insertion site of the user; generate, from the glucose measurements, one or more features for the first time periods of the multiple time windows; detect, from the one or more features for the first time periods of the multiple time windows, a pattern in the glucose measurements in the first time periods of the multiple time windows; determine a behavior modification feedback to improve glucose levels corresponding to the pattern; generate a user interface including the behavior modification feedback; and cause the user interface to be displayed on the display device.

In some aspects, the techniques described herein relate to a computing device, wherein each time window includes one day, the multiple time windows include multiple days, and each of the multiple time periods includes a different multi-hour period of time during a day.

In some aspects, the techniques described herein relate to a computing device, wherein to detect a pattern is to determine that criteria for a feature of the one or more features is not satisfied.

In some aspects, the techniques described herein relate to a computing device, wherein the pattern is one of multiple patterns detected in the glucose measurements, each of the multiple patterns being one of the one or more features for which corresponding criteria is not satisfied, and the instructions further cause the processor to normalize the multiple patterns to generate a size for each of the multiple patterns.

In some aspects, the techniques described herein relate to a computing device, wherein the instructions further cause the processor to generate a numeric value for the user based on the glucose measurements or the one or more features, and customize the behavior modification feedback to the user by including at least one numeric value in the behavior modification feedback.

In some aspects, the techniques described herein relate to a device including: a display device; a behavior library including multiple behavior modification feedback; a glucose measurement collection module, implemented at least in part in hardware, to obtain, from a glucose sensor of a continuous glucose level monitoring system and for each time window of multiple time windows, glucose measurements measured for a user for a first time period of multiple time periods of the time window, the glucose sensor being inserted at an insertion site of the user; a feature determination module, implemented at least in part in hardware, to generate, from the glucose measurements, one or more features for the first time periods of the multiple time windows; a pattern detection module, implemented at least in part in hardware, to detect, from the one or more features for the first time periods of the multiple time windows, a pattern in the glucose measurements in the first time periods of the multiple time windows; a behavior modification selection module, implemented at least in part in hardware, to determine a behavior modification feedback from the behavior library to improve glucose levels corresponding to the pattern, to generate a user interface including the behavior modification feedback, and to cause the user interface to be displayed on the display device.

In some aspects, the techniques described herein relate to a device, wherein each time window includes one day, the multiple time windows include multiple days, and each of the multiple time periods includes a different multi-hour period of time during a day.

In some aspects, the techniques described herein relate to a device, wherein to detect a pattern is to determine that criteria for a feature of the one or more features is not satisfied.

In some aspects, the techniques described herein relate to a device, wherein the pattern is one of multiple patterns detected in the glucose measurements, each of the multiple patterns being one of the one or more features for which corresponding criteria is not satisfied, and the device further includes a normalization module, implemented at least in part in hardware, to normalize the multiple patterns to generate a size for each of the multiple patterns.

In some aspects, the techniques described herein relate to a computing device, further including a behavior modification feedback customization module, implemented at least in part in hardware, to generate a numeric value for the user based on the glucose measurements or the one or more features, and customize the behavior modification feedback to the user by including at least one numeric value in the behavior modification feedback.

Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method implemented in a continuous glucose level monitoring system, the method comprising:
obtaining, from a glucose sensor of the continuous glucose level monitoring system and for each time window of multiple time windows, glucose measurements measured for a user for a first time period of multiple time periods of the time window, the glucose sensor being inserted at an insertion site of the user;
generating, from the glucose measurements, one or more features for the first time periods of the multiple time windows;
detecting, from the one or more features for the first time periods of the multiple time windows, a pattern in the glucose measurements in the first time periods of the multiple time windows;
determining a behavior modification feedback to improve glucose levels corresponding to the pattern;
generating a user interface including the behavior modification feedback; and
causing the user interface to be displayed.

2. The method of claim 1, wherein each time window comprises one day, the multiple time windows comprise multiple days, and each of the multiple time periods comprises a different multi-hour period of time during a day.

3. The method of claim 2, wherein the multiple time periods includes an overnight time period, an after breakfast time period, an after lunch time period, and an after dinner time period.

4. The method of claim 2, further comprising receiving user input specifying, for each of the multiple time periods, the multi-hour period of time during the day for the time period.

5. The method of claim 2, further comprising automatically learning, by a machine learning system, at least one of the multi-hour periods of time of the day.

6. The method of claim 1, wherein each time window comprises one week, the multiple time windows comprise multiple weeks, and each of the multiple time periods comprises a different day in a week.

7. The method of claim 1, wherein the detecting a pattern comprises determining that criteria for a feature of the one or more features is not satisfied.

8. The method of claim 7, wherein the pattern is one of multiple patterns detected in the glucose measurements, each of the multiple patterns being one of the one or more features for which corresponding criteria is not satisfied, the method further comprising normalizing the multiple patterns to generate a size for each of the multiple patterns.

9. The method of claim 8, wherein the determining the behavior modification feedback includes selecting behavior modification feedback corresponding to one of the multiple patterns having a largest size.

10. The method of claim 1, further comprising:
generating a numeric value for the user based on the glucose measurements or the one or more features; and
customizing the behavior modification feedback to the user by including at least one numeric value in the behavior modification feedback.

11. The method of claim 1, wherein the pattern is one of multiple patterns detected in the glucose measurements, and the determining the behavior modification feedback includes selecting behavior modification feedback mapped to by one of multiple topics that is mapped to by one of the multiple patterns.

12. The method of claim 1, wherein the pattern is one of multiple patterns in the glucose measurements, and the determining the behavior modification feedback includes selecting behavior modification feedback corresponding to one of the multiple patterns.

13. The method of claim 12, further comprising receiving activity data for the user from an activity tracker, and wherein the selecting behavior modification feedback includes not selecting behavior modification feedback indicating to perform activity that the activity data indicates the user is already performing

14. The method of claim 1, further comprising:
subsequently determining whether the behavior modification in the behavior modification feedback was performed by the user; and
providing additional feedback congratulating the user in response to determining that the behavior modification in the behavior modification feedback was performed by the user.

15. A computing device comprising:
a processor;
a display device; and
computer-readable storage media having stored thereon multiple instructions of an application that, responsive to execution by the processor, cause the processor to:
obtain, from a glucose sensor of a continuous glucose level monitoring system and for each time window of multiple time windows, glucose measurements measured for a user for a first time period of multiple time periods of the time window, the glucose sensor being inserted at an insertion site of the user;
generate, from the glucose measurements, one or more features for the first time periods of the multiple time windows;
detect, from the one or more features for the first time periods of the multiple time windows, a pattern in the glucose measurements in the first time periods of the multiple time windows;
determine a behavior modification feedback to improve glucose levels corresponding to the pattern;
generate a user interface including the behavior modification feedback; and
cause the user interface to be displayed on the display device.

16. A device comprising:
a display device;
a behavior library including multiple behavior modification feedback;
a glucose measurement collection module, implemented at least in part in hardware, to obtain, from a glucose sensor of a continuous glucose level monitoring system and for each time window of multiple time windows, glucose measurements measured for a user for a first time period of multiple time periods of the time window, the glucose sensor being inserted at an insertion site of the user;
a feature determination module, implemented at least in part in hardware, to generate, from the glucose measurements, one or more features for the first time periods of the multiple time windows;
a pattern detection module, implemented at least in part in hardware, to detect, from the one or more features for the first time periods of the multiple time windows, a pattern in the glucose measurements in the first time periods of the multiple time windows; and
a behavior modification selection module, implemented at least in part in hardware, to determine a behavior modification feedback from the behavior library to improve glucose levels corresponding to the pattern, to generate a user interface including the behavior modification feedback, and to cause the user interface to be displayed on the display device.

17. The device of claim 16, wherein each time window comprises one day, the multiple time windows comprise multiple days, and each of the multiple time periods comprises a different multi-hour period of time during a day.

18. The device of claim 16, wherein to detect a pattern is to determine that criteria for a feature of the one or more features is not satisfied.

19. The device of claim 16, wherein the pattern is one of multiple patterns detected in the glucose measurements, each of the multiple patterns being one of the one or more features for which corresponding criteria is not satisfied, and the device further comprising a normalization module, implemented at least in part in hardware, to normalize the multiple patterns to generate a size for each of the multiple patterns.

20. The device of claim 16, further comprising a behavior modification feedback customization module, implemented at least in part in hardware, to generate a numeric value for the user based on the glucose measurements or the one or more features, and customize the behavior modification feedback to the user by including at least one numeric value in the behavior modification feedback.
